(19) United States
(12) Patent Application Publication
(10) Pub. No.: US 2004/0197294 A1
(43) Pub. Date: Oct. 7, 2004

(54) Cosmetic and/or pharmaceutical preparations

(76) Inventors: Werner Seipel, Hilden (DE); Lars Zander, Duesseldorf (DE); Thomas Albers, Duesseldorf (DE)

Correspondence Address: COGNIS CORPORATION, PATENT DEPARTMENT, 300 BROOKSIDE AVENUE, AMBLER, PA, 19002, US

(21) Appl. No.: 10/492,438

(22) PCT Filed: Oct. 4, 2003
(86) PCT No.: PCT/EP02/11118

(30) Foreign Application Priority Data
Oct. 13, 2001 (DE) 10150728.3

(51) Int. Cl. A61K 7/075; A61K 7/08

(52) U.S. Cl. 424/70.21; 424/70.22

(57) ABSTRACT

A cosmetic and/or pharmaceutical composition containing: (a) a glycerol monoalkyl ether; (b) a surfactant selected from the group consisting of an anionic surfactant, a nonionic surfactant, a cationic surfactant, an amphoteric surfactant, a zwitterionic surfactant, and mixtures thereof; (c) optionally, up to about 10% by weight of a fatty alcohol; and (d) optionally, water.

### FIELD OF THE INVENTION

This invention relates to surfactant preparations containing glyceride monoalkylethers and to the use of glyceride monoalkylethers as lipid layer enhancers in surfactant preparations.

### PRIOR ART

Preparations used to clean and care for the human skin and hair generally contain one or more surfactants, more particularly based on anionic or amphoteric surfactants. Since the use of surfactants on their own would excessively dry out the skin and hair, lipid layer enhancers are generally added to such preparations. However, surfactant-containing formulations on the one hand are intended to have a cleaning efect, but on the other hand are nevertheless intended to leave a lipid-layer-enhancing protective film behind on the skin. With rinse-off formulations in particular, the difficulty is that lipid-layer-enhancing substances are also rinsed off and are unable adequately to develop their effect. The replacement of unwanted fatty constituents on the skin, which are intended to undergo the cleaning process, by lipid-layer-enhancing constituents which are intended to remain on the skin or to be incorporated in the upper layers of the skin is subject to a delicate balance.

EP 0 547 727 B1 discloses glycerol monoalkyl ethers as lipid layer enhancers for skin disinfectants. Since the formulations in question are leave-on formulations, glycerol monoethers may be used irrespective of their lipophilia. Short-chain glyceryl monoalkyl ethers in low concentrations are preferably used in these formulations in order to exploit their good alcohol solubility and additional antimicrobial activity. Nevertheless, the sebum content clearly decreases with time after application and it is only repeated application that leaves the skin with a higher fat content than is the case with conventional lipid-layer-enhancing disinfectants.

Surfactant formulations often contain glyceryl monoalkyl esters as lipid layer enhancers, cf. for example German patents DE 41 39 935 C2 and DE 19543633 C2. These compositions contain relatively large quantities of fatty acid monoglycerides because a significant proportion is rinsed off again by the cleaning properties. U.S. Pat. No. 4,690,818 describes ethoxylated glyceryl ethers as moisturizers for skin and hair in cosmetic cleaning preparations. In this case, however, the moisturizing effect is based on a water-binding effect and not on a lipid-layer-enhacing effect which keeps the water in the skin for much longer by occlusion.

Accordingly, the problem addressed by the present invention was to provide rinse-off formulations which would combine a good cleaning effect with an improved lipid-layer-enhancing effect in relation to the prior art and which would leave the skin with a pleasant feeling, would show high dermatological compatibility and would be easy to produce. The lipid layer enhancers used in the preparations would not reduce their viscosity.

### DESCRIPTION OF THE INVENTION

The present invention relates to cosmetic and/or pharmaceutical preparations containing

(a) 0.1 to 5% by weight glycerol monoalkyl ethers,

(b) 0.1 to 90% by weight anionic and/or nonionic and/or cationic and/or amphoteric and/or zwitterionic surfactants and optionally

(c) 0.1 to 10% by weight fatty alcohols,

and to the use of glycerol monoalkylethers as lipid layer enhancers in surfactant-containing formulations.

It has surprisingly been found that the combination of glycerol monoalkylethers with surfactants leads to rinse-off formulations which combine a very good cleaning effect with optimal lipid-layer-enhancing properties. The mixtures leave the skin with a pleasant feeling and show particularly good dermatological compatibility. When used in hair care preparations, they produce an improvement in combability. The processing behavior of the mixtures can be improved by the addition of fatty alcohols without any shift in the balance between cleaning effect and lipid-layer-enhancing effect, despite the change in the lipophilic/hydrophilic balance.

Glycerol Monoalkyl Ethers

The glycerol monoalkyl ethers selected are known substances which may be produced by the relevant methods of preparative organic chemistry. Mixtures of mono-, di- and triethers are generally formed during the production process. Accordingly, the used as glycerol monoalkylethers in the present invention are fatty acid mono-/di-/triglyceride ethers which have a mono content of at least 80% by weight, preferably at least 90% by weight and more particularly at least 95% by weight. They correspond to formula (I):

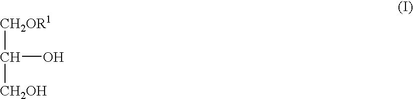

in which R¹ is an unbranched or branched alkyl and/or alkenyl group containing 6 to 18 carbon atoms, preferably 8 to 16 carbon atoms and more particularly 12 to 14 carbon atoms.

The glyceryl monoalkylethers are used in the preparations according to the invention in quantities of 0.1 to 10% by weight, preferably in quantities of 1 to 5% by weight and more particularly in quantities of 2 to 3% by weight.

Surfactants

Suitable surfactants are nonionic, anionic, cationic and/or amphoteric or zwitterionic surfactants which are normally present in the preparations in quantities of about 1 to 90, preferably 5 to 70 and more particularly 10 to 50% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl(ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Alkyl and/or Alkenyl Oligoglycosides

Alkyl and alkenyl oligoglycosides which are used as partiularly preferred surfactants are known nonionic surfactants which corresponding to formula (II):

R²O—[G]ₚ (II)

in which R² is an alkyl and/or alkenyl group containing 4 to 22 carbon atoms, G is a sugar unit containing 5 or 6 carbon atoms and p is a number of 1 to 10. They may be obtained by the relevant methods of preparative organic chemistry, for example by acid-catalyzed acetalization of glucose with fatty alcohols.

The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in general formula (II) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.4 are preferred from the applicational point of view.

The alkyl or alkenyl group R² may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₋₂ alcohol as an impurity, and also alkyl oligoglucosides based on technical C_{9/11} oxoalcohols (DP=1 to 3) are preferred. In addition, the alkyl or alkenyl radical R⁹ may also be derived from primary alcohols containing 12 to 22 and preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligo-glucosides based on hydrogenated C_{12/14} cocoalcohol with a DP of 1 to 3 are preferred. Alkyl and/or alkenyl oligoglycosides are used in quantities of 1 to 90% by weight, preferably in quantities of 5 to 70% by weight and more particularly in quantities of 10 to 50% by weight in the preparations according to the invention.

Fatty Alcohols

Fatty alcohols are understood to be primary aliphatic alcohols corresponding to formula (III):

R³OH (III)

where R³ is an aliphatic, linear or branched hydrocarbon radical containing 6 to 22 carbon atoms and 0 and/or 1, 2 or 3 double bonds. Typical examples are caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxo synthesis and as monomer fraction in the dimerization of unsaturated fatty alcohols. Preferred fatty alcohols are technical C₁₂₋₁₈ fatty alcohols such as, for example, coconut oil, palm oil, palm kernel oil or tallow fatty alcohol. In order not to disturb the balance between cleaning effect and lipid-layer-enhancing effect, fatty alcohols containing 12 to 14 carbon atoms are particularly preferred.

These fatty alcohols are used in the preparations according to the invention in quantities of 0.1 to 10% by weight, preferably in quantities of 0.5 to 5% by weight and more particularly in quantities of 0.8 to 3% by weight.

The fatty alcohols have the advantage that the incorporation of the glycerol monoethers can be considerably improved.

Commercial Applications

The preparations according to the invention are distinguished by a high cleaning capacity and by excellent lipid-layer-enhancing properties. They show high dermatological compatibility, are liquid and pumpable and, where fatty alcohols are added, can be produced without heat in the form of a compound. This compound (lipid-layer-enhancing compound) contains 50 to 90% by weight glycerol monoalkylethers and 10 to 50% by weight fatty alcohols, preferably 60 to 80% by weight glycerol monoalkylethers and 20 to 35% by weight fatty alcohols, more preferably 65 to 75% by weight glycerol monoalkylethers and 25 to 40% by weight fatty alcohols and most preferably 70% by weight glycerol monoalkylethers and 30% by weight fatty alcohols. The fatty alcohols used have chains with a length of 6 to 22, preferably 12 to 18 and more particularly 12 to 14 carbon atoms.

The advantage of compounding lies in cold processing. In contrast to direct incorporation at at least 60° C., the compound is added to a formulation without heating. The compound itself is mixed at 40 to 45° C.

Accordingly, the present invention also relates to the use of glycerol monoalkylethers and combinations of glycerol monoalkylethers with fatty alcohols as lipid layer enhancers in surfactant-containing formulations, for example for hair and body care.

Embodiments of the cosmetic and/or pharmaceutical preparations according to the invention contain

a) 0.1 to 10% by weight glycerol monoalkylethers,

b) 1 to 90% by weight anionic and/or nonionic and/or cationic and/or amphoteric and/or zwitterionic surfactants and

c) 0 to 10% by weight fatty alcohols,

preferably

a) 1 to 5% by weight glycerol monoalkylethers,

b) 5 to 70% by weight anionic and/or nonionic and/or cationic and/or amphoteric and/or zwitterionic surfactants and

c) 0 to 5% by weight fatty alcohols,

more preferably

a) 2 to 3% by weight glycerol monoalkylethers,

b) 10 to 50% by weight anionic and/or nonionic and/or cationic and/or amphoteric and/or zwitterionic surfactants and

c) 0 to 3% by weight fatty alcohols,

most preferably

a) 0.1 to 10% by weight glycerol monoalkylethers,

b) 1 to 90% by weight anionic and/or nonionic and/or cationic and/or amphoteric and/or zwitterionic surfactants and

c) 0.5 to 5% by weight fatty alcohols and

in one most particularly preferred embodiment

a) 1 to 5% by weight glycerol monoalkylethers,

b) 5 to 70% by weight anionic and/or nonionic and/or cationic and/or amphoteric and/or zwitterionic surfactants and

c) 0.8 to 3% by weight fatty alcohols.

The cosmetic and/or pharmaceutical preparations mentioned, such as for example hair shampoos, shower baths, foam baths, washing lotions . . . and the like, may contain emulsifiers, pearlizing waxes, consistency factors, thickeners, polymers, silicone compounds, lecithins, phospholipids, biogenic agents, antioxidants, antidandruff agents, film formers, hydrotropes, solubilizers, preservatives, perfume oils, dyes and the like as further auxiliaries and additives.

Emulsifiers

Suitable emulsifiers are, for example, nonionic surfactants from at least one of the following groups:

products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and alkylamines containing 8 to 22 carbon atoms in the alkyl group;

addition products of 15 to 60 mol ethylene oxide onto castor oil and/or hydrogenated castor oil;

partial esters of glycerol and sorbitan with unsaturated, linear or saturated, branched fatty acids containing 12 to 22 carbon atoms and/or hydroxycarboxylic acids containing 3 to 18 carbon atoms and addition products thereof onto 1 to 30 mol ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof onto 1 to 30 mol ethylene oxide;

mixed esters of pentaerythritol, fatty acids, citric acid and fatty alcohol according to DE 1165574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethylene glycol-30 Dipolyhydroxystearate;

polymer emulsifiers, for example Pemulen types (TR-1, TR-2) of Goodrich;

polyalkylene glycols and

glycerol carbonate.

>Ethylene Oxide Addition Products

The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as lipid layer enhancers for cosmetic formulations from DE 2024051 PS.

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide with the partial glycerides mentioned are also suitable.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Anionic Emulsifiers

Typical anionic emulsifiers are aliphatic fatty acids containing 12 to 22 carbon atoms, such as for example palmitic acid, stearic acid or behenic acid, and dicarboxylic acids containing 12 to 22 carbon atoms, such as azelaic or sebacic acid for example.

Amphoteric and Cationic Emulsifiers

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable consistency factors are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Polymers

Suitable cationic polymers are, for example, cationic cellulose derivatives such as, for example, the quaternized hydroxyethyl cellulose obtainable from Amerchol under the name of Polymer JR 400®), cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosm. Toil., 108, 95 (1993).

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Biogenic Agents

Biogenic agents in the context of the invention are, for example, tocopherol, tocopherol acetate, tocopherol palmitate, ascorbic acid, (deoxy)ribonucleic acid and fragmentation products thereof, α-glucans, retinol, bisabolol, allantoin, phytantriol, panthenol, AHA acids, amino acids, ceramides, pseudoceramides, essential oils, plant extracts, for example prunus extract and bambara nut extract, and vitamin complexes.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxyphenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol, >sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetik-verordnung (“Cosmetics Directive”).

Perfume Oils and Aromas

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat. Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes as listed, for example, in the publication “Kosmetische Färbemittel” of the Farbstoffkommission der Deutschen Forschungs-gemeinschaft, Verlag Chemie, Weinheim, 1984, pages 81 to 106. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS(C.I. 69800) and madder lake (C.I. 58000). Luminol may also be present as a luminescent dye. These dyes are normally used in concentrations of 0.001 to 0.1% by weight, based on the mixture as a whole.

### EXAMPLES

### I. Application Examples—Dematological Compatibility—TEWL

Results

Dermatological compatibility was evaluated by determining the transepidermal water loss (TEWL) in a pig's epidermis. To this end, defined pieces of skin were treated with the various test solutions for 30 minutes at 40° C. and the TEWL value was gravimetrically determined. The test solutions were mixtures of 17% by weight Plantapon® PS 10 (sodium lauryl ether sulfate and Lauryl Glycoside, Cognis Dusseldorf) with 1.5% by weight fatty acid partial glycerides or glycerol ethers (see Table 1) in preservative-containing water (WAS 10% by weight). The pH was adjusted to 5.5 with citric acid. The results are set out in Table 1. The lower the value, the better the dermatological compatibility.

TABLE 1
Transepidermal water loss

| | | TEWL |
|---|---|---|
| | | [%-rel] = |
| | | [treated/ |
| | Fatty acid partial glycerides | untreated * |
| Example | (Mono/(di)glyceride ratio) | 100] − 100% |
| 1 | Glycerol mono/dicapryl ether (65:35) | 19.0 |
| 2 | Glycerol monocapryl ether (>90) | 5.0 |
| 3 | Glycerol mono/dicaprylate (65:35) | 18.0 |
| 4 | Glycerol monocaprylate (>90) | 6.5 |
| 5 | Stearic acid mono/diglyceride (65:35) | 21.5 |
| 6 | Stearic acid monoglyceride (>90) | 15.0 |
| 7 | Isostearic acid mono/diglyceride (65:35) | 22.0 |
| 8 | Isostearic acid monoglyceride (>90) | 18.5 |
| 9 | Lauric acid mono/diglyceride (65:35) | 20.5 |
| 10 | Lauric acid monoglyceride (>90) | 10.5 |
| C1 | No partial glyceride, only 17% by | 22.0 |
| | weight Plantapon PS 10 | |

The Examples and the Comparison Example (C1) clearly show that glycerol monothers with 90% purity and monoglycerides with a monoglyceride content of at least 90% by weight produce a far lower transepidermal water loss than the comparison mixtures consisting of only 65% by weight monoether and 35% by weight diether and hence show significantly better dermatological compatibility.

### II. Application Examples—Performance Data

Four conventional formulations (C1 to C4) were compared in their properties with Examples R5 to R8 according to the invention.

The irritation potential was determined by the HET-CAM Test, as described in “Der HET-CAM-Test”, Euro Cosmetics 11/12-99, pp. 29-33, Köszegi, Dunja et al.

Lipid-layer-enhancing behavior was evaluated by determining the wet combability of treated hair tresses. To this end, the tresses were medium-bolded before the zero measurement. After a contact time of 5 minutes, the test formulations (1 g/1 g hair) were rinsed for 1 minute under standard conditions (38° C., 1 liter/minute). The measurement was carried out on 20 hair tresses.

TABLE 2
Comparison of conventional formulations (C1 to C4) with formulations according to
the invention containing glycerol monocapryl ether (R5 to R8) (percentages in % by
weight active substance)

| Trade Name | INCI Name | C1 | C2 | C3 | C4 | R5 | R6 | R7 | R8 |
|---|---|---|---|---|---|---|---|---|---|
| Texapon ® N 70 | Sodium Laureth Sulfate | 17.0 | 17.0 | 17.0 | 17.0 | 17.0 | 17.0 | 17.0 | 17.0 |
| Dehyton ® PK | Cocamidopropyl Betaine | 7.5 | 7.5 | 7.5 | 7.5 | 7.5 | 7.5 | 7.5 | 7.5 |
| 45 | | | | | | | | | |
| | Glycerol monocapryl | — | — | — | — | — | — | 3.0 | 5.0 |
| | ether | | | | | | | | |
| | Glycerol monocapryl | — | — | — | — | 1.0 | 3.0 | — | — |
| | ether compound | | | | | | | | |
| Cetiol ® HE | Polyoxyethylene | 2 | — | — | — | — | — | — | — |
| | Glyceryl | | | | | | | | |
| | Monococoate | | | | | | | | |
| Castor oil | | — | 1* | — | — | — | — | — | — |
| Soybean oil | | — | — | 1* | — | — | — | — | — |
| Sodium chloride | | 3.1*** | — | — | 2.2 | 1.5 | 1.3 | 1.0 | 0.95 |
| Citric acid | | for pH adjustment | | | | | | | |
| Preserved water | | to 100.0 | | | | | | | |
| pH value | | 5.5 | | | | | | | |
| Viscosity | | 3200*** | — | — | 6000 | 6800 | 8600 | 9400 | 11300 |
| [mPa * s]**** | | | | | | | | | |
| Appearance | | Clear | Cloudy | Cloudy | Clear | Clear | Clear | Clear | Clear |
| Skin feel | | − | + | + | −− | ++ | +++ | +++ | +++ |
| HET-CAM Q | | 1.9 | − | − | >2 | 1.3 | 1.0 | 1.3 | 1.5 |
| Wet combability | | −− | −− | −− | −−− | ++ | ++ | ++ | +++ |

*clear only with a solvent or viscous with a thickener
**clear solubility limit
***viscosity limit
****viscosity measured with a Brookfield RVT, Spindle 4 at 10 r.p.m./RT = 23° C.

The glycerol monocapryl ether compound consists of 70% by weight glycerol monocapryl ether and 30% by weight Lorol® spezial A (INCI: Lauryl Alcohol)

### II. Formulation Examples—Incorporation Limit Before Clouding

TABLE 3a
Direct incorporation in a surfactant formultion (percentages as
% by weight; water + preservative to 100%)

| Trade Name | INCI name | R1 | R2 | R3 | R4 | R5 | R6 | R7 |
|---|---|---|---|---|---|---|---|---|
| Texapon ® N 70 | Sodium Laureth Sulfate | 17.0 | 17.0 | 17.0 | 17.0 | 17.0 | 17.0 | 17.0 |
| Dehyton ® PK | Cocamidopropyl Betaine | 7.5 | 7.5 | 7.5 | 7.5 | 7.5 | 7.5 | 7.5 |
| 45 | | | | | | | | |
| | Glycerol monocapryl | 1.0 | 2.0 | 3.0 | 4.0 | 5.0 | 6.0 | 10.0 |
| | ether | | | | | | | |

| Water | to 100 | | | | | | |
|---|---|---|---|---|---|---|---|
| Appearance | Clear | Clear | Clear | Clear | Clear | Cloudy | Cloudy |

TABLE 3b
Incorporation via a compound (percentages as
% by weight; water + preservative to 100%)

| Trade Name | INCI Name | R1 | R2 | R3 | R4 |
|---|---|---|---|---|---|
| Texapon ® N 70 | Sodium Laureth | 17.0 | 17.0 | 17.0 | 17.0 |
| | Sulfate | | | | |
| Dehyton ® PK | Cocamidopropyl | 7.5 | 7.5 | 7.5 | 7.5 |
| 45 | Betaine | | | | |
| | Glycerol monocapryl | 1.0 | 2.0 | 3.0 | 4.0 |
| | ether compound | | | | |

| Water | to 100 | | | |
|---|---|---|---|---|
| Appearance | Clear | Clear | Clear | Cloudy |

The glycerol monocapryl ether compound consists of 70% by weight glycerol monocapryl ether and 30% by weight Lorol ® spezial A (INCI: Lauryl Alcohol).

TABLE 4
Application Examples (percentages as % by weight; water + preservative to 100%)

| Preparation | Component | CTFA Name | Content % |
|---|---|---|---|
| Hair rinse | Lanette ® O | Cetearyl Alcohol | 4.0 |
| | Cutina ® GMS-V | Glycerylstearate | 0.5 |
| | Dehyquart ® L80 | Dicocoylethyl hydroxyethylmonium | 2.5 |
| | | Methosulfate (and) Propylenglycol | |
| | | Hydroxypropyl Guar | 0.5 |
| | Jaguar ® HP 105 | | 2.5 |
| | Glycerol monocapryl | | |
| | ether | | |
| | Water | | to 100.0 |
| Shower bath | Texapon ® K 14 S | Sodium Myreth Sulfate | 35.0 |
| | Plantacare ® 818 UP | Coco Glucoside | 6.0 |
| | Dehyton ® PK 45 | Cocamidopropyl Betaine | 8.0 |
| | Lorol ® spezial | Lauryl Alcohol | 0.9 |
| | Glycerol monocapryl | | 2.1 |
| | ether | | |
| | Water | | to 100.0 |
| “2 in 1” | Texapon ® N70 | Sodium Laureth Sulfate | 15.0 |
| Shower bath | Plantacare ® 818 UP | Coco Glucoside | 5.0 |
| | Dehyton ® PK 45 | Cocoamidopropyl Betaine | 8.0 |
| | Luviquat ® Care | Polyquaternium 44 | 3.0 |
| | Gluadin ® W 40 | Hydrolyzed Wheat Protein | 2.0 |
| | Glycerol monocapryl | | 2.1 |
| | ether | | |
| | Lorol ® spezial | Lauryl Alcohol | 0.9 |
| | Water | | to 100.0 |
| Shampoo | Texapon ® NSO | Sodium Laureth Sulfate | 34.0 |
| | Plantacare ® 818 UP | Coco Glucoside | 3.0 |
| | Dehyton ® MC | Sodium Cocoamphoacetate | 5.0 |
| | Gluadin ® WQ | Laurdimonium Hydroxypropyl | 2.0 |
| | | Hydrolyzed Wheat Protein | |
| | Glycerol monocapryl | | 1.4 |
| | ether | | |
| | Lorol ® spezial | Lauryl Alcohol | 0.6 |
| | Water | | to 100.0 |

1-10 (cancelled).

11. A cosmetic and/or pharmaceutical composition comprising:
(a) a glycerol monoalkyl ether;
(b) a surfactant selected from the group consisting of an anionic surfactant, a nonionic surfactant, a cationic surfactant, an amphoteric surfactant, a zwitterionic surfactant, and mixtures thereof;
(c) optionally, up to about 10% by weight of a fatty alcohol; and
(d) optionally, water.

12. The composition of claim 11 wherein (a) is present in the composition in an amount of from about 0.1 to 10% by weight, based on the weight of the composition.

13. The composition of claim 11 wherein (a) is present in the composition in an amount of from about 1 to 5% by weight, based on the weight of the composition.

14. The composition of claim 11 wherein (b) is present in the composition in an amount of from about 0.1 to 90% by weight, based on the weight of the composition.

15. The composition of claim 11 wherein (b) is present in the composition in an amount of from about 5 to 70% by weight, based on the weight of the composition.

16. The composition of claim 11 wherein (c) is present in the composition in an amount of from about 0.5 to 5% by weight, based on the weight of the composition.

17. The composition of claim 11 wherein (c) is present in the composition in an amount of from about 0.8 to 3% by weight, based on the weight of the composition.

18. The composition of claim 16 wherein (c) is a C₁₀₋₁₈ fatty alcohol.

19. The composition of claim 16 wherein (c) is a C₁₂₋₁₄ fatty alcohol.

20. A composition comprising:
(a) from about 50 to 90% by weight of a glycerol monoalkyl ether; and
(b) from about 10 to 50% by weight of a fatty alcohol, and wherein the composition is capable of being cold processed.

21. A process for treating human skin and/or hair comprising contacting the skin and/or hair with a composition containing:
(a) a glycerol monoalkyl ether;
(b) a surfactant selected from the group consisting of an anionic surfactant, a nonionic surfactant, a cationic surfactant, an amphoteric surfactant, a zwitterionic surfactant, and mixtures thereof;
(c) optionally, up to about 10% by weight of a fatty alcohol; and
(d) optionally, water.

22. The process of claim 21 wherein (a) is present in the composition in an amount of from about 0.1 to 10% by weight, based on the weight of the composition.

23. The process of claim 21 wherein (a) is present in the composition in an amount of from about 1 to 5% by weight, based on the weight of the composition.

24. The process of claim 21 wherein (b) is present in the composition in an amount of from about 0.1 to 90% by weight, based on the weight of the composition.

25. The process of claim 21 wherein (b) is present in the composition in an amount of from about 5 to 70% by weight, based on the weight of the composition.

26. The process of claim 21 wherein (c) is present in the composition in an amount of from about 0.5 to 5% by weight, based on the weight of the composition.

27. The process of claim 21 wherein (c) is present in the composition in an amount of from about 0.8 to 3% by weight, based on the weight of the composition.

28. The process of claim 26 wherein (c) is a C₁₀₋₁₈ fatty alcohol.

29. The process of claim 26 wherein (c) is a C₁₂₋₁₄ fatty alcohol.
